(19) United States
(12) Patent Application Publication
(10) Pub. No.: US 2009/0012436 A1
(43) Pub. Date: Jan. 8, 2009

(54) ORTHOSIS AND TREATMENT METHOD

(75) Inventors: Gerd Lanfermann, Aachen (DE); Richard Daniel Willmann, Siegburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/279,230

(22) PCT Filed: Feb. 9, 2007
(86) PCT No.: PCT/IB2007/050423

§ 371 Date: Aug. 13, 2008

(30) Foreign Application Priority Data
Feb. 17, 2006 (EP) 06110083.0

(51) Int. Cl. A61F 5/01; A61N 1/36; A61F 7/00

(52) U.S. Cl. 602/2; 607/3; 607/96

(57) ABSTRACT

Orthosis, configured to be worn by a patient, particularly to treat a medical disorder of the patient, the orthosis (1) comprising: a rigid support device (2), for example a rigid exoskeleton or a rigid frame, to support the patient and/or correct a posture of the patient during use; a system (11) to provide electrical treatment, for example a FES system to provide functional electrical stimulation; and a system (20) to provide thermal treatment.

The invention relates to an orthosis, as well as to a method to treat a patient, for example to treat spasticity.

Orthoses as such are known from the prior art. Generally, an orthosis is applied to firmly support a part of a patient, for example a limb. A specific type of orthosis is the so-called FES orthosis. FES stands for ‘functional electrical stimulation’. The FES orthosis can provide electrical stimulation of, for example, a muscle of a patient, for example to treat a certain spastic disorder.

The known orthosis system comprises a hand-forearm orthosis containing an array of electrodes, connected to an electronic microprocessor-controlled unit. This known orthosis system can provide neuromuscular stimulation, for therapeutic treatment. The system comprises a control unit, which sends pulses of low-level electrical current to the electrodes integrated into the orthosis. The known unit initiates phased-patterned stimulation sequences to activate the muscles in selected order for functional or therapeutic exercise of the hand. Functional modes and therapeutic treatment programs can be programmed into a microprocessor.

The known FES orthosis system has several disadvantages. Particularly, a respective FES treatment is usually not suitable to be applied continuously over a long time, since such a treatment can lead to muscle fatigue.

It is an object of the present invention to alleviate the above-mentioned problems. Particularly, the invention aims to provide improved means and an improved method to treat a patient, for example to treat spasticity and/or other muscle disorders According to the invention, this object is achieved by the features of claim 1.

Advantageously, the orthosis comprises:
a rigid support device, for example a rigid exoskeleton or a rigid frame, to support the patient and/or correct a posture of the patient during use;
a system to provide electrical treatment, for example a FES system to provide functional electrical stimulation; and
a system to provide thermal treatment.

The orthosis, provided by the invention, can be used to apply both a FES treatment and a thermal treatment to the patient, which can lead to considerably improved treatment results. For example, during use, when the orthosis is carried by the patient and supports part of the patient and/or corrects a posture of the patient, the orthosis can treat the patient for a relatively long time. The orthosis can provide FES treatment and thermal treatment a suitable number of times, in a suitable order.

Besides, an embodiment of the invention provides a method to treat a patient, wherein the patient is provided with an orthosis according to the invention, such that the orthosis supports the patient and/or corrects a posture of the patient, wherein part of the patient, for example a muscle part, is electrically treated and thermally treated by the orthosis. For example, the method can involve treatment of a spastic disorder, and/or an other muscle disorder. This can provide the above-mentioned advantages, and can particularly lead to an improved treatment of the patient, for example to overcome spasticy in an effective manner.

In a further embodiment, a computer program, or software, is provided, with instructions arranged for carrying out or controlling a method according to the invention when said program is executed by a computer, or hardware respectively.

A basic idea behind the present invention is, that a FES orthosis is equipped with a thermal treatment system, for example a heating or cooling element, to provide and coordinate additional muscle relieve therapies. Thus, for example, spasticity can be treated much more effectively until the present, nobody came upon the simple idea that a FES orthosis can be advantageously provided with a thermal treatment system.

Further advantageous embodiments of the invention are described in the dependent claims. These and other aspects of the invention will be apparent from and elucidated with reference to the embodiments described hereafter.

The invention will now be described in more detail on the basis of exemplary embodiments shown in the accompanying drawing, wherein:

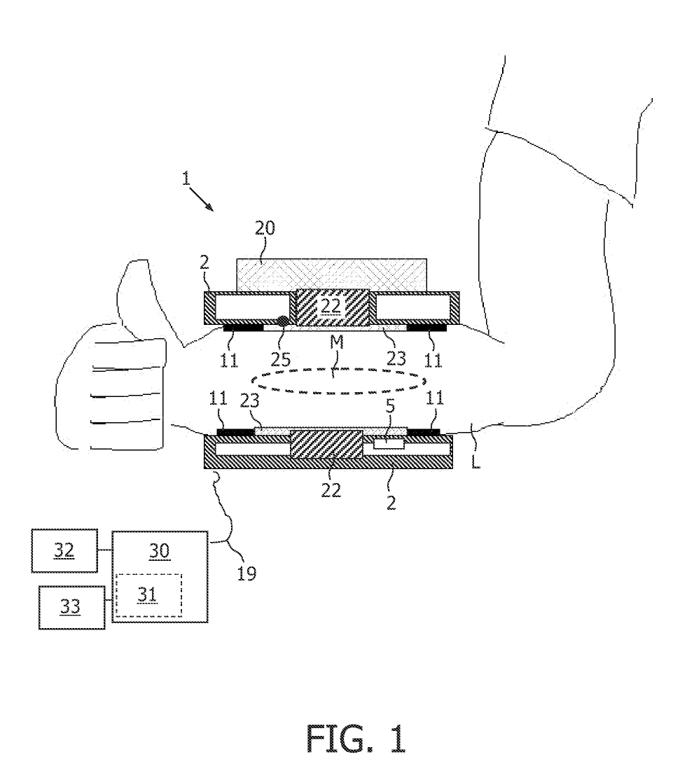

FIG. 1 schematically depicts an embodiment of the invention, during use, wherein the orthosis is depicted in a cross-section;

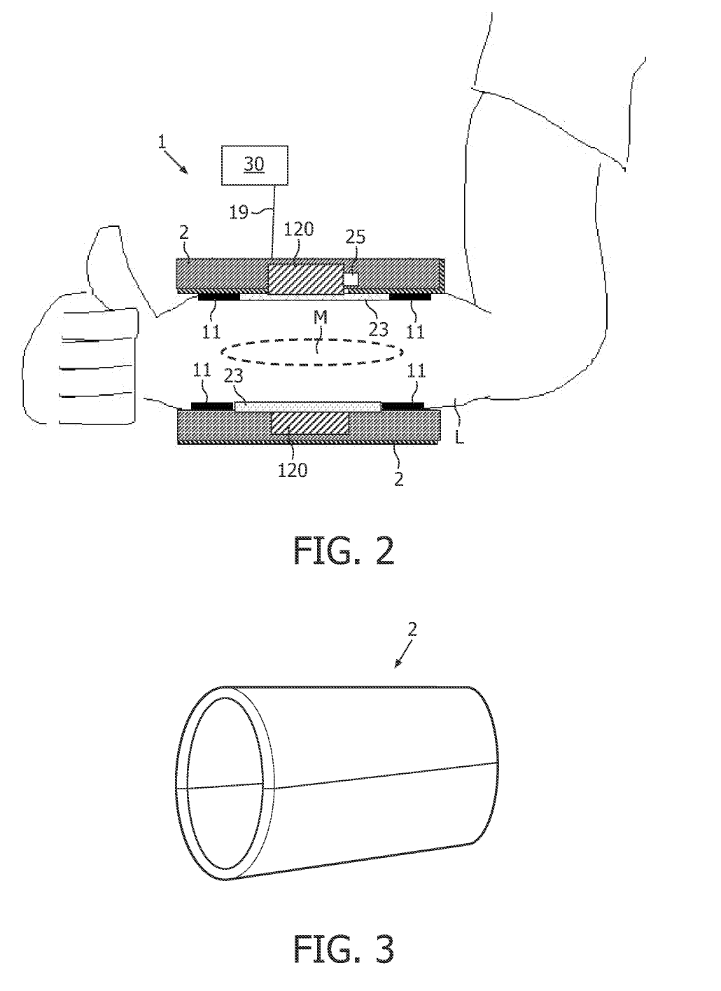

FIG. 2 schematically depicts an alternative embodiment of the invention, during use, wherein the orthosis is depicted in a cross-section;

FIG. 3 schematically depicts a perspective view of an support part according to an embodiment;

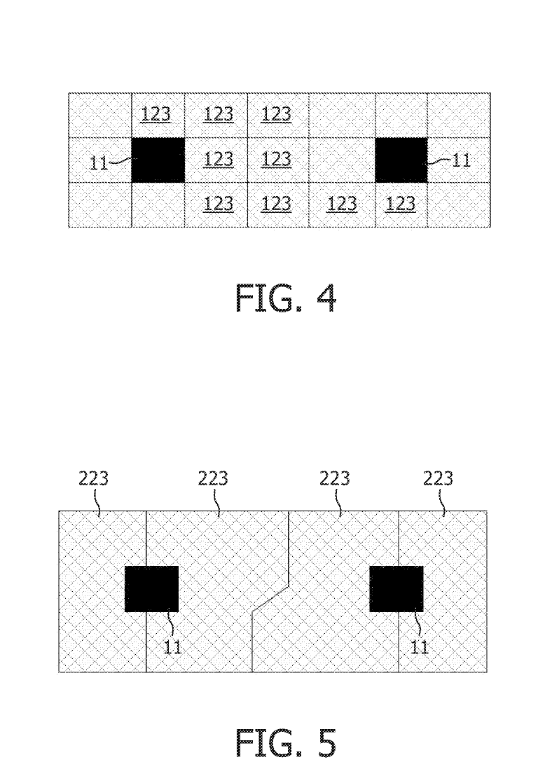

FIG. 4 schematically shows a further embodiment of the invention, depicting part of an inner surface of the orthosis; and

FIG. 5 schematically shows an other further embodiment of the invention, depicting part of an inner surface of the orthosis.

FIG. 1 schematically shows an embodiment of a FES orthosis, or orthotic support device, 1, during use. The orthosis 1 is configured to be worn by a patient, particularly to treat a medical disorder of the patient, for example to treat a spastic disorder of a limb L. A muscle, to be treated, is schematically indicated in FIG. 1 by dashed lines M. In FIG. 1, the orthosis is substantially carried by the forearm L of the user. On the other hand, the orthosis can be configured to be carried by a different user limb to be treated, for example a leg, and/or an other user part, for example a torso.

The orthosis 1 comprises a rigid support device 2, for example a rigid exoskeleton, a rigid frame or an external brace, configured to support one or more parts of the patient and/or correct a posture of the patient. Such a support device can be configured in various ways, as will be clear to the skilled person. The support device 2 can be an exo-skeleton, which can attach to a user's limb L to support, stabilize, and correct the posture, or to monitor and assist in the execution of movements during rehabilitation. For example, the support device 2 can be configured to hold one patient part in a substantially fixed position with respect to an other patient part, for example to firmly hold and stabilize a joint. The support device 2 can be configured to be attached or connected to the different parts of the patient, for example to a hand (or wrist) and the respective fore-arm to position the hand (or wrist) and fore-arm firmly with respect to each other. Also, for example, the support device 2 can be configured to counteract certain muscle contractions of the respective patient part L. Thus, also, the orthosis can be arranged to counteract movement of a limb or part of the patient.

For example, the mentioned exo-skeleton can include one or more substantially rigid, or substantially non-flexible, frame members 2 (which are depicted schematically in FIGS. 1 and 2), for example rods, bars, plates and/or other frame members, which can apply a desired supporting force or pressure to the part L of the patient that is to be treated and/or to be fixed in a desired position. The frame members 2 can be made of various materials, can have various shapes, as will be clear to the skilled person. For example, the frame members 2 can comprise or consist of one or more rigid plastics, metals, alloys and/or other materials, rigid composite materials, fiber reinforced compositions and/or a combination of such and other materials.

The mentioned exo-skeleton may e.g. come as a tube 2, which can be opened e.g. along a long side, and which can hold the arm fixed, or support the arm, inside. For example, in an embodiment, FIG. 1 shows a cut through such a system, with the arm and arm muscles in the center, substantially surrounded by the rigid structure 2 of the orthosis. FIG. 3 shows a perspective sketch of a skeleton part of such a system, wherein the support part 4 is substantially tube-shaped.

Besides, in the present embodiment, the orthosis is provided with a system to provide electrical treatment of the limb L, for example a FES system to provide functional electrical stimulation of the muscle M. For example, the rigid support device 2 can be provided with one or more electrodes 11, connected or connectable to a power source 30, 32, particularly to apply high-frequency electric signals to trigger muscle activity of the patient during use. Each of the electrodes 11 can be configured in various ways. Preferably, each electrode 11 is configured to make electrical contact with the skin of the user, to apply electrical treatment.

Also, the orthosis 1 can comprise or be connectable to a controller 30, to control the electrical treatment system and/or the thermal treatment system. For example, the mentioned power source 32 can be part of the controller 30, and/or can be an external power source. Preferably, the application of the mentioned electric signals to the electrodes is controllable by the controller 30. The controller 30, power source 32 and electrodes 11 can be communicatively connected to each other in various ways, for example by suitable wiring 19, and/or wirelessly, as will be clear to the skilled person.

As an example, the controller 30 can include suitable hardware, computer means, a personal computer, a portable controller, a PDA (personal digital assistant), micro-electronics, a memory, a timer 33, and/or other controller means, as will be clear to the skilled person. The controller 30 can be at least partly provided by software. For example, a computer program can be provided, or software, with instructions arranged for controlling at least part of the treatment when said program is executed by a computer, or hardware respectively.

Preferably, treatment parameters, relating to a treatment to be carried out, can be stored in the controller 30. Besides, the controller 30 can be provided with, or can be coupled to, a suitable user interface 31, through which the user and/or a physiotherapist can activate, adjust and/or operate the controller. For example, the user interface 31 can be configured to allow a user or operator to adjust mentioned treatment parameters, and/or to program one or more functional modes and therapeutic treatment programs.

Also, in a further embodiment, a (personalized) treatment protocol can be either stored or downloaded, for example utilizing a portable memory device, a Memory stick, floppy disk, using secure internet access to medical professional and/or in a different manner. The controller 30 can be configured to access and apply the mentioned treatment protocol, to provide a desired treatment.

Besides, the orthosis 1 can comprise or be connectable to at least a first sensor 5, which can be configured to measure or determine an effect of the electrical treatment. Such a first sensor 5 can be configured in various ways. For example, such a sensor 5 can include one or more strain or pressure gauges, which can be configured to determine muscle activity or muscle contractions of/in the respective part (limb L) of the patient. Also, such a sensor can be configured to derive the effects of the electric treatment physiologically by deriving an EMG (electromyogram). Preferably, such sensor 5 is connected or connectable to the mentioned controller 30, for example to provide measurement results to the controller and/or to be powered by the controller.

Besides, advantageously, the orthosis 1 is provided with a system to provide thermal treatment of the limb L. This thermal treatment system can be configured in various ways. For example, the orthosis can be configured to carry a thermal treatment device 20. In a further embodiment, the thermal treatment device 20 can be configured to exchange heat with a respective user's limb L to thermally treat the limb L. For example, the thermal treatment device can comprise a suitable thermal treatment agent.

In a further embodiment, the thermal treatment system includes a cooling system, wherein the treatment device 20 is a cooling device, and comprises a cooling agent, for example a freezable cooling agent, during use. The cooling system can provide cooling, particularly to cool one or more muscles M of a part of the patient, which carries the orthosis 1 during use. For example, the cooling system can be configured to provide a cooling of the limb's skin surface to 15-20 degrees Celsius, or to lower temperatures, during a desired treatment period. In the present embodiment, such a cooling can allow the orthosis 1 to cool the limb L during a thermal treatment session of an overall treatment, for example to relax muscle contractions.

The mentioned thermal treatment device 20 can be configured or operate, for example, similar to the cooling device of the depilation system described in U.S. Pat. No. 6,406,483 B1, which patent is incorporated in the present application by reference. For example, the cooling agent can comprise or consist of a mixture comprising water, a coloring agent, and a preserving agent.

In the present embodiment, the thermal treatment system can also be configured to provide a certain heating of the patient, for example to warm one or more muscles M of a part L of the patient which carries the orthosis 1 during use. In that case, for example, the thermal treatment device 20 can be provided with a heat able heating agent instead of a cooling agent. The thermal treatment system can then be configured or operate, for example, similar to the heating device of the depilation system described in U.S. Pat. No. 6,689,143 B1, which patent is incorporated in the present application. For example, the heating agent can be a compound having a eutectic composition.

For example, the thermal treatment system can comprise a heating member 20 for generating a rise in temperature of the skin. Also, for example, the thermal treating system can comprise an electrical heating system (for example comprising one or more electric heaters, and/or Peltier elements 120, see below), to electrically heat at least part of, for example, a limb L (or the skin thereof) to be treated.

In the embodiment of FIG. 1, the thermal treatment device comprises a housing or casing 20 in which a certain quantity of a thermal treatment agent (not depicted) is or can be contained. Preferably, the housing 20 is removable coupled to the support device 2, so that the housing 20 can be cooled or heated separately. For example, in case a cooling treatment is desired, the housing can be cooled in a separate freezing device, for example in a freezer compartment of a refrigerator or a home freezer.

As is shown in FIG. 1, in the present embodiment, the orthosis can be provided with one or more skin contact areas 23, to provide thermal contact with the patient during use, particularly to receive heat from the patient and/or to supply heat to the patient. A heat conductor 22 can be provided, to conduct heat between a skin contact part or area 23 and the mentioned housing 20.

In the present embodiment, a mentioned skin contact area 23 is configured to extend opposite the muscle part M of the limb, along the user's skin, during use. In the drawing, the skin contact area 23 is shown to substantially surround part of the arm of the user. Preferably, the skin contact area 23 can provide a peripheral thermal treatment of the limb L. Besides, for example, the skin contact area 23 can be configured to abut various sides of the user's skin, and/or to substantially surround the limb L. The orthosis 1 can be provided with one or more skin contact areas in various ways. FIGS. 4-5 show other embodiments, with different arrangements of skin contact areas, as will be explained below.

The orthosis can be provided with one or more of the mentioned heat conductor 22. Each heat conductor 22 can be fixed to the support device 2, and/or be made in one part with the support device 2. Alternatively, the heat conductor 22 can be connected to, or be part of the housing 20. The heat conductor 22 can be made of one or more materials having a relatively high thermal conductivity, for example one or more metals, aluminum and/or different materials. A mentioned skin contact part or area 23 of the heat treatment system can be a surface part of the heat conductor 22, or be connected or connectable to the heat conductor 22. The skin contact part area 23 can be of the made material as the heat conductor 22, of different material. In the present embodiment, during use, the housing 20 can be in a suitable thermal contact with the heat conductor 22, for example to receive heat from the patient's skin that abuts the mentioned skin contact part 23 to provide cooling treatment of a muscle part M, or to apply heat to the skin in a heating treatment to heat the muscle part M.

FIG. 2 shows an alternative embodiment. In FIG. 2, the thermal treatment system including at least one Peltier element 120. The Peltier element 120 can be controlled by the controller 30, to cool or heat the limb L of the patient. For example, the skin contact area 23 can be actively cooled down, or alternatively warmed up, by means of the one or more Peltier elements 120. Besides, in an embodiment, the orthosis can be configured to apply one or more such Peltier elements 120 in conjunction of a mentioned thermal treatment agent.

In a preferred embodiment, as is depicted in FIGS. 1 and 2, the skin contact area 23 of the thermal treatment system can be located and/or extend between mentioned electrodes 11 of the electrical treatment system. In the present embodiment, the electrodes 11 are arranged next to the skin contact area 23 for thermal treatment. For example, the orthosis can be configured to apply electrical treatment and thermal treatment at least to substantially the same muscle part M of the patient during use, for example to hand muscles M which are located in the fore-arm. In a further embodiment, the electrodes 11 and the skin contact area 23 of the thermal treatment system are arranged to make contact with different skin surfaces at the same time, when the orthosis 1 is being worn by the patient. Thus, interference between heat treatment and electrical treatment can be prevented. For example, in this manner, electrodes 11 can be kept relatively free of undesired condensing water, for the case that a cooling treatment was or is performed as well.

Also, the one or more electrodes 11 and the one or more skin contact areas 23 can be separate from each other, or at least partially overlap, and/or can be at least partially integrated with each other.

Besides, the orthosis 1 can comprise one or more second, thermal, sensors 25, for example to detect a result of a mentioned thermal treatment and/or to monitor the thermal treatment. The monitoring can involve, for example, timing the time period during which a certain thermal treatment is still present, and/or detecting an amount of thermal treatment. The monitoring can also involve detecting a certain end of a thermal treatment, for example in case the at least one thermal sensor 25 detects a predetermined threshold temperature which relates to an effective ending of the thermal treatment. A thermal sensor 25 can be provided, for example, to measure temperature at or near a skin contact area 23 of the thermal treatment system, and/or the measure the temperature of the skin of the user. Besides, for example, one or more such temperature sensing means 25 can be configured to measure a temperature of part of the orthosis 1, to measure a temperature of a mentioned thermal treatment agent, or to measure a temperature of the environment of the orthosis 1. During use, the controller 30 preferably receives sensor signals from such one or more thermal sensors 25.

In one embodiment, the orthosis 1 can be configured to automatically alternate electrical treatment and thermal treatment. The alternating of the treatments can be, for example, abruptly, or gradually such as via ramping. Also, for example, the orthosis 1 can be configured to apply the electrical treatment and thermal treatment at least partially at the same time. Herein, the controller 30 can control both the electrical treatment system and/or the thermal treatment system, and can carry out a desired treatment schedule. For example, the controller can be a mentioned timer 33, to automatically start, adjust and/or stop the electrical FES treatment at one or more predetermined times during operation. For example, a FES session can be automatically stopped after a predetermined time period, to prevent muscle fatigue.

Besides, a mentioned electrical heating system or a mentioned Peltier element 120 can be timer controllable by the controller 30. Thus, the thermal treatment can be activated, adjusted and/or deactivated at desired, suitable times. For example, the amount of thermal treatment, provided by such an electrical thermal treatment system 120, can be controlled and adjusted in a well-defined manner. Also, in this way, the thermal treatment system can provide multiple heating or cooling sessions, for example at least one session before and at least one session after each FES treatment of the electrical treatment system. Besides, in the embodiment of FIG. 2, the one or more Peltier elements 120 can also provide thermal treatment during a mentioned electrical FES treatment.

The controller 30 is preferably configured to control the electrical treatment system and/or the thermal treatment system, using one or more parameters, such, that one or more parameters concerning the electrical treatment and one or more parameters concerning the thermal treatment are mutually dependent. Thus, an optimum or well-balanced treatment may be reached, leading to improved results.

As is shown in FIG. 1, during use, the orthosis 1 can treat the patient, wherein the limb of the patient is provided with the orthosis 1, such that the orthosis 1 supports the patient and/or corrects a posture of the patient.

For example, in case a cooling of the muscle part M is desired, the housing 20 can first be externally cooled in a freezer, to cool a thermal treatment agent. Then, the housing 20 can be attached to the orthosis 1, to provide the cooling treatment of the muscle M. The thermal conductor 22 can then ensure good heat transport between cooling agent in the housing 20 and the contact area for the skin 23, to cool the skin and to thermally treat the muscle M.

Alternatively, the thermal treatment system of the orthosis can be used to provide a certain (active) heating of the limb L, for example by providing a heating agent instead of a cooling agent, as is mentioned above.

The use of the embodiment of FIG. 2 can be similar to the use of the embodiment of FIG. 1. In that case, the one or more Peltier elements 120 can be activated to provide the thermal treatment, instead of (or—if applicable—in addition to) the use of a cooling or heating agent.

At one ore more desired moments, also, the electrical treatment system can provide a certain FES treatment of the muscle M, via the electrodes 11. In each case, preferably, the overall treatment, which includes both the thermal treatment and the FES treatment, can be substantially automated, and can be under the control of the controller 30. The muscle part M of the limb L can be electrically treated and thermally treated by the orthosis 1, for example to treat a spastic disorder. The thermal treatment and electrical treatment can be applied at least partially at the same time, if desired.

Also, the thermal treatment and electrical treatment can be timed, and/or controlled automatically, by a mentioned timer 33. The thermal treatment can be started, by controller 30, a predetermined amount of time before the electrical treatment is started. Besides, the thermal treatment can be automatically started or maintained during a predetermined amount of time, after electrical treatment has stopped. Also, the length of the electrical treatment is preferably under timer control.

Besides, as is mentioned above, one or more parameters concerning the electrical treatment and one or more parameters concerning the thermal treatment can be mutually dependent. The method can comprise measuring or determining an effect of the electrical treatment, and using the result of such a measurement or determination to control or adjust the thermal treatment.

Preferably, a controlling of the treatment systems is dependent of results provided by one or more of the above-mentioned sensors 5, 25. For example, the controller 30 can be configured to start, change or increase a FES treatment, provided via the electrical treatment system, in case a mentioned thermal sensor 25 has detected a certain decrease of a mentioned thermal treatment, provided by the thermal treatment system, and/or in case a sensor signal of the thermal sensor 25 passes a certain predetermined threshold value. Similarly, the controller 30 can be configured to start, change or increase a thermal treatment, provided via the thermal treatment system, in case a mentioned first sensor 5 has detected a certain decrease of a mentioned electrical treatment, provided by the electrical treatment system, and/or in case a sensor signal of the first sensor 5 passes a certain predetermined threshold value. Also, the treatment, provided by the orthosis 1, can be controllable in a different manner and order.

FIG. 4 shows an alternative embodiment, which differs from the embodiments shown in FIGS. 1-2 in that the orthosis is provided with multiple skin contact areas 123, to provide a heating/cooling. FIG. 4 provides a view towards part of an surface of the orthosis, which surface is faced towards the skin of a user during use. FIG. 4 depicts mentioned electrodes 11 as well as a number of the skin contact areas 123. In the present embodiment, the electrodes 11 are each surrounded by a plurality of the skin contact areas 123. Preferably, the several skin contact areas 123 can be heated and/or cooled independently. For example, the shape of these skin contact areas 123 or units may be designed according to match a location and extension of one or more targeted muscles M. For example, to this aim, the orthosis can be provided with independent thermal sources for each skin contact area 123. The control of each skin contact area 123 e.g. is done depending on the stimulation through the individual electrodes 11.

FIG. 5 shows an other alternative embodiment, which differs from the embodiments shown in FIGS. 1-2 in that the orthosis is provided with an array of skin contact areas 223, to provide a heating/cooling. FIG. 5 also provides a view towards part of the inner surface of the orthosis, and depicts mentioned electrodes 11 as well as the array of skin contact areas 223. In the present embodiment, the skin contact areas 223 are substantially parallel to each other. In the present embodiment, the electrodes 11 are each surrounded by two of the skin contact areas 223 of the array. Preferably, the skin contact areas 223 of the array can be heated and/or cooled independently. Also, for example, to this aim, the orthosis can be provided with independent thermal sources for each skin contact area 223. Again, the control of each skin contact area 223 e.g. can be done depending on the stimulation through the individual electrodes 11. With such a system, the individual muscle location and extension could easily be matched by turning on/off the respective skin contact areas.

The present orthosis 1 can provide a very effective treatment of, for example, spasticity. The additional cooling of a spastic limb L can provide additional and extended improvement. The orthosis 1 can be used for home rehabilitation.

Spasticity is a disorder of the body's motor system in which certain muscles are continuously contracted. This contraction causes stiffness or tightness of the muscles and may interfere with gait, movement, and speech. Spasticity is usually caused by damage to the portion of the brain or spinal cord that controls voluntary movement. For example, the electrical treatment can involve applying high-frequency electric currents through the electrodes 11, to trigger muscle activity. Herein, for example, pulses of low-level electrical current can be sent to the electrodes 11, to initiate phased-patterned stimulation sequences to activate the muscle(s) M in selected order for functional or therapeutic exercise of the hand. Triggering the antagonistic muscle opens up the spastic contraction. The thermal treatment system can cool the muscle M, to relax the contraction. The present orthosis can provide integration of a cooling system into an FES orthosis, to provide advantages of both cooling and FES, for example whenever a stimulation session is initiated by the patient or a physiotherapist.

The one or more cooling areas 23 can make contact to the skin of the patient, and can be activated, when a FES session is initiated by the patient or the physiotherapist.

The present embodiment 1 can be carried for a long time, continuously, by the patient. Also, the orthosis 1 can treat the limb L over a long time period, by combining thermal treatment with FES treatment, for example in appropriate order. Herein, undesired muscle fatigue can be prevented well. Also, the application of separate cooling cushions first, before the orthosis 1 is being applied to the limb, can be prevented. Such separate cooling cushion might interfere with FES treatment. Application of separate cooling cushions could, for example, make the skin wet due to condensing water, hindering a subsequent FES treatment.

The present orthosis 1 can provide an easy way for a patient for an automated combination therapy. The integration of a cooling device into the FES orthosis 1 can offer the following advantages, some of which are also mentioned above. Firstly, cooling and electrical stimulation can be applied at the same time: whenever the FES device is used, the muscle is cooled as well. Also, cooling can be integrated into a common treatment protocol. FES and cooling can automatically be scheduled, via the controller 30 and/or timer 33, without removing one of the two systems (FES , cooling). Cooling can be provided before the FES treatment: e.g. 2 hours before to FES phase is scheduled. The muscle is cooled in preparation of the FES session. Furthermore, cooling can be provided after the FES treatment: after the electrical stimulation has been applied to the arm, die muscle is cooled to extend the benefit. In a further embodiment, cooling can be automatically provided between the FES sessions. Besides, for example, cooling parameters (temperature, duration) can be defined based on the FES parameters. E.g. a reduced FES strength can be compensated by increased cooling with respect to a lower temperature and extended cooling duration. The cooling parameters may be defined based on the response of the muscle to FES. The effects of the FES may be determined mechanically, e.g. the ability of the muscle to contract (through strain gauges 5 around the arm). Besides, in an embodiment, FES parameters may set depending on cooling parameters.

Although the illustrative embodiments of the present invention have been described in greater detail with reference to the accompanying drawings, it will be understood that the invention is not limited to those embodiments. Various changes or modifications may be effected by one skilled in the art without departing from the scope or the spirit of the invention as defined in the claims.

It is to be understood that in the present application, the term “comprising” does not exclude other elements or steps. Also, each of the terms “a” and “an” does not exclude a plurality. Any reference sign(s) in the claims shall not be construed as limiting the scope of the claims.

1. Orthosis, configured to be worn by a patient, particularly to treat a medical disorder of the patient, the orthosis (1) comprising:
a rigid support device (2), to support the patient and/or correct a posture of the patient during use;
a system (11) to provide electrical treatment; and
a system (20) to provide thermal treatment.

2. Orthosis according claim 1, configured to provide electrical treatment and thermal treatment at least to substantially a same part (L) of the patient during use.

3. Orthosis according to claim 1, configured to automatically alternate electrical treatment and thermal treatment.

4. Orthosis according to claim 1, configured to apply the electrical treatment and thermal treatment at least partially at same time.

5. Orthosis according to claim 1, comprising or connectable to a controller (30), to control the electrical treatment system and/or the thermal treatment system.

6. Orthosis according to claim 5, wherein the controller is provided with a timer (33) to automatically start, adjust and/or stop the electrical treatment and/or thermal treatment at one or more predetermined times during operation.

7. Orthosis according to claim 5, wherein the controller (30) is configured to control the electrical treatment system and/or the thermal treatment system, using one or more parameters, such, that one or more parameters concerning the electrical treatment and one or more parameters concerning the thermal treatment are mutually dependent.

8. Orthosis according to claim 1, further comprising or being connectable to at least one sensor (5) configured to measure or determine an effect of the electrical treatment, wherein preferably a controlling of the thermal treatment system is dependent of results provided by the at least one sensor (5).

9. Orthosis according to claim 1, further comprising or being connectable to at least one temperature sensor (25) configured to monitor the thermal treatment and/or determine an effect of the thermal treatment, wherein preferably a controlling of the electrical treatment system is dependent of results provided by the at least one temperature sensor (25).

10. Orthosis according to claim 1, wherein the rigid support device (2) is provided with one or more electrodes (11), connected or connectable to a power source, particularly to apply high-frequency electric signals to trigger muscle activity of the patient during use.

11. Orthosis according to claim 1, provided with one or more skin contact areas (23) to provide thermal contact with the patient during use, particularly to receive heat from the patient and/or to supply heat to the patient.

12. Orthosis according to claim 1, configured to carry a thermal treatment device (20), the thermal treatment device (20) comprising a thermal treatment agent.

13. Orthosis according to claim 1, the thermal treatment system including at least one Peltier element (120).

14. Method to treat a patient, wherein the patient is provided with an orthosis according to claim 1, such that the orthosis supports the patient and/or corrects a posture of the patient, wherein the method includes the steps of electrically treating and thermally treating a part of the patient by the orthosis.

15. Method according claim 14, wherein the method includes treating a spastic disorder.

16. Method according to claim 14, wherein the thermal treatment and electrical treatment are applied at least partially at a same time.

17. Method according to claim 14, wherein the thermal treatment and electrical treatment are controlled automatically by a timer.

18. Method according to claim 14, wherein the thermal treatment is being started a predetermined amount of time before the electrical treatment is started

19. Method according to claim 14, wherein the thermal treatment is automatically started or maintained during a predetermined amount of time, after electrical treatment has stopped

20. Method according to claim 14, wherein one or more parameters concerning the electrical treatment and one or more parameters concerning the thermal treatment are mutually dependent.

21. Method according to claim 14, comprising measuring or determining an effect of the electrical treatment, and using the result of such a measurement or determination to control or adjust the thermal treatment.

22. Method according to claim 14, involving timing a time period during which a certain thermal treatment is still present, detecting an amount of thermal treatment, and/or detecting a certain end of a thermal treatment.

23. A computer program, or software, with instructions arranged for carrying out or controlling a method according to claim 14 when said program is executed by a computer, or hardware respectively.
